(19) United States
(12) Patent Application Publication
(10) Pub. No.: US 2026/0306811 A1
(43) Pub. Date: Oct. 8, 2026

(54) MEDICAL MOBILE ROBOT FOR DIGESTIVE SYSTEM, METHOD OF USING SAME, AND TREATMENT METHOD USING MEDICAL MOBILE ROBOT

(71) Applicant: KOREA INSTITUTE OF SCIENCE AND TECHNOLOGY, Seoul (KR)

(72) Inventors: Junghwan BYUN, Seoul (KR); Metin SITTI, Istanbul (TR); Yoosoo YANG, Gyeonggi-do (KR); Siyeon JANG, Stuttgart (DE)

(73) Assignee: KOREA INSTITUTE OF SCIENCE AND TECHNOLOGY, Seoul (KR)

(21) Appl. No.: 19/635,198

(22) Filed: Mar. 31, 2026

(30) Foreign Application Priority Data
Apr. 2, 2025 (KR) 1020250042581

(51) Int. Cl. A61M 31/00; A61B 34/00; A61B 34/30

(52) U.S. Cl. CPC A61M 31/002; A61B 34/73; A61B 2034/303; A61M 2205/0244; A61M 2205/0294; A61M 2205/3515; A61M 2210/1042

(57) ABSTRACT

Proposed are a medical mobile robot for a digestive system, a method of using the same, and a treatment method using the medical mobile robot, wherein the medical mobile robot includes a magnetic body part configured to enable shape change and movement within the digestive system due to magnetic force, a piezoelectric transducer coupled to the magnetic body part to convert an ultrasonic signal into an electrical signal, an electronic component coupled to the magnetic body part to at least modulate or process the electrical signal generated by the piezoelectric transducer.

### CROSS REFERENCE TO RELATED APPLICATION

The present application claims priority to Korean Patent Application No. 10-2025-0042581, filed Apr. 2, 2025, the entire contents of which are incorporated herein for all purposes by this reference.

### BACKGROUND OF THE INVENTION

### Field of the Invention

The present disclosure relates to medical equipment, a method of using the same, and a medical treatment method and, more particularly, to a medical mobile robot orally administered into a digestive system of a target body, a method of using the same, and a treatment method using the medical mobile robot.

This invention was supported by the Nano & Material Technology Development Program through the National Research Foundation of Korea (NRF), funded by the Korean government (Ministry of Science and ICT) (Grant No. RS-2024-00407155).

### Description of the Related Art

In general, targeted anticancer therapy is administered by injecting anticancer drugs or treatment agents into a human body through intravascular injection such as intravenous injection. However, in this case, there is a problem that the accuracy and efficiency of drug delivery are significantly reduced. The probability that a drug injected into the bloodstream will reach a cancer tumor is less than 1%, and even though the drug reaches the tumor site, the drug must penetrate into the tumor through diffusion, so the probability that the drug will enter the inside of the tumor may be less than 0.001%. To increase treatment efficacy, the concentration and amount of a treatment agent injected into the bloodstream have to be increased, but this is limited owing to lethal doses and side effects. The overdose of the drug in the bloodstream can cause the side effects at non-target sites. Meanwhile, in the case of oral administration, efficiency with which orally administered drug enters the bloodstream is less than 10%, so the drug delivery efficiency may be more than 1/10th lower than that of the intravascular injection.

Recently, a cell death mechanism using copper ions has been discovered, which is called cuproptosis. There are many attempts to treat cancer by using such a cell death mechanism. However, most of these attempts deliver nanoparticles bound to the copper ions via an intravenous injection. The nanoparticles can reach sites in the vicinity of cancer cells (a tumor), and the nanoparticles (compounds) can be decomposed in the corresponding site in a predetermined manner, so as to emit the copper ions. However, even in this case, for the same reasons as described above, the drug dosage may be very limited and the drug delivery efficiency may also be extremely low.

Therefore, it is required to develop new approaches and the related technologies that may overcome the limitations and problems of existing targeted anticancer therapy using intravascular injection.

### SUMMARY OF THE INVENTION

An objective of the present disclosure for solving the technical problems is to provide a medical mobile robot for a digestive system, the medical mobile robot being inserted into the digestive system of a living body to treat a disease or perform a medical procedure, and being able to dramatically improve the delivery efficiency, delivery accuracy, and the resulting treatment effects of a treatment substance to a target site.

In addition, another objective of the present disclosure for solving the technical problems is to provide a medical mobile robot being able to easily move (walk) in a desired direction and to a desired position and perform a given movement through shape change in an environment inside a digestive system of a living body, i.e., in an environment that is sticky, is humid (wet), has high friction, contains digestive fluid, mucus, or the like, and has movement (e.g., movement of the stomach).

In addition, yet another objective of the present disclosure for solving the technical problems is to provide a medical mobile robot being able to significantly improve treatment effect of diseases such as digestive system cancer by positioning itself accurately at a target site within a digestive system of a living body to release a large amount of a treatment substance and dramatically improving the penetration efficiency of the treatment substance into the target.

In addition, yet another objective of the present disclosure for solving the technical problems is to provide a method of using the above-described medical mobile robot.

In addition, still another objective of the present disclosure for solving the technical problems is to provide a treatment method using the above-described medical mobile robot and a medical method related to the above-described medical mobile robot.

The problems to be solved by the present disclosure are not limited to the problems mentioned above, and other problems not mentioned herein may be understood by those skilled in the art from the description below.

According to one exemplary embodiment of the present disclosure, there is provided a medical mobile robot for a digestive system and being inserted into the digestive system of a living body to treat a disease or perform a medical procedure, the medical mobile robot including: a magnetic body part configured to enable shape change and movement within the digestive system due to magnetic force; a piezoelectric transducer coupled to the magnetic body part to convert an ultrasonic signal into an electrical signal; an electronic component coupled to the magnetic body part to at least modulate or process the electrical signal generated by the piezoelectric transducer; and solid-state metal source members coupled to the magnetic body part and capable of emitting metal ions in response to an electrical input from the electronic component.

The magnetic body part may include: an elastic polymer matrix; and a plurality of magnetic particles inserted into the elastic polymer matrix.

The magnetic body part may have a radial structure provided with three or more leg parts extending outward from a central part thereof when viewed from above in a fundamental form.

The magnetic body part may be provided with a plurality of leg parts extending outward from a central part thereof in a fundamental form, and the magnetic body part may have a radial magnetization structure in which a magnetization direction of each of the plurality of leg parts is aligned outward from the central part.

The electronic component may include a signal converter for converting an AC voltage signal into a DC voltage signal.

Each metal source member may be configured to emit the metal ions by electrochemical dissolution.

Each metal source member may be a bulk-type metal body.

Each metal source member may satisfy at least one size condition among conditions of a length in a range of 10 μm to 5 mm, a width in a range of 10 μm to 1 mm, and a thickness in a range of 100 nm to 1 mm.

The metal ions may include Cu²⁺ ions.

The magnetic body part may be provided with a plurality of leg parts extending outward from a central part thereof in a fundamental form, and the metal source members may be arranged to be combined with the plurality of respective leg parts.

An electrode wiring element connected to each metal source member may be provided on each of the plurality of leg parts, and the electrode wiring element may have a winding shape.

The electronic component and each metal source member may be configured to generate electric fields while the metal ions are emitted, and effective diffusivity of the metal ions may increase by the electric fields.

The medical mobile robot may further include a bioadhesive layer for increasing adhesive strength to a given site of the living body within the digestive system.

The bioadhesive layer may include: an elastic material layer having a surface provided with a plurality of protrusions thereon; and an adhesive coating layer placed on the elastic material layer so as to cover the plurality of protrusions.

The medical mobile robot may have a length in a range of 0.5 to 20 mm and a width in a range of 0.5 to 20 mm.

The medical mobile robot may be a cancer treatment robot for treating digestive system cancer.

The medical mobile robot may be configured to hold a tumor site within the digestive system with the magnetic body part.

According to another exemplary embodiment of the present disclosure, there is provided a method of using a medical mobile robot described above, the method including: inserting the medical mobile robot into a digestive system of a living body; and operating the medical mobile robot within the digestive system of the living body.

The medical mobile robot may be inserted into the living body in a method of oral administration in a state of being placed in a capsule, and at least a part of the capsule may be dissolved or the medical mobile robot comes out of the capsule within the predetermined digestive system.

The operating of the medical mobile robot may include: applying a magnetic field to the medical mobile robot; and applying an ultrasonic signal to the medical mobile robot.

The operating of the medical mobile robot may include: changing a shape or moving a position of the medical mobile robot within the digestive system; and emitting metal ions from each metal source member.

The medical mobile robot may be operated while capturing an image of the digestive system.

According to exemplary embodiments of the present disclosure, a medical mobile robot for a digestive system and a method of using the same may be implemented, the medical mobile robot being inserted into the digestive system of a living body to treat a disease or perform a medical procedure, and being able to dramatically improve the delivery efficiency and delivery accuracy of the treatment substance to a target site.

In addition, according to the exemplary embodiments of the present disclosure, the medical mobile robot and the method of using the same may be implemented, the medical mobile robot being able to easily move (walk) in a desired direction and to a desired position and perform a given movement through shape change in an environment inside a digestive system of a living body, i.e., in an environment that is sticky, is humid (wet), has high friction, contains digestive fluid, mucus, or the like, and has movement (e.g., movement of the stomach).

In addition, according to the exemplary embodiments of the present disclosure, the medical mobile robot and the method of using the same may be implemented, the medical mobile robot being able to significantly improve the treatment effect of diseases such as digestive system cancer by positioning itself accurately at a target site within a digestive system of a living body to release a large amount of a treatment substance and to dramatically improving the penetration efficiency of the treatment substance into the target.

Using a treatment method (a medical method) according to the exemplary embodiments of the present disclosure enables to improve the delivery efficiency of a treatment substance (a drug) to a target within a digestive system to almost 100%, significantly increase the treatment effect with a large amount of a treatment substance, and suppress or prevent the problem of side effects.

### BRIEF DESCRIPTION OF THE DRAWINGS

FIG. 1 is an exploded perspective view illustrating a medical mobile robot for a digestive system according to one exemplary embodiment of the present disclosure.

FIG. 2 is a perspective view illustrating the medical mobile robot for the digestive system according to one exemplary embodiment of the present disclosure.

FIG. 3 is a perspective view illustrating an example of a case in which a magnetic field is applied to the medical mobile robot for the digestive system to change the shape of a magnetic body part according to one exemplary embodiment of the present disclosure.

FIG. 4 is a photographic image showing the medical mobile robot for the digestive system and manufactured according to one exemplary embodiment of the present disclosure.

FIG. 5 is a view showing photographic images of the lower and upper surfaces of the medical mobile robot for the digestive system and manufactured according to one exemplary embodiment of the present disclosure.

FIG. 6 is a photographic image showing a case in which the medical mobile robot for the digestive system is positioned within a gastrointestinal tract of a living body according to one exemplary embodiment of the present disclosure.

FIG. 7 is a photographic image showing a case in which only the magnetic body part is positioned within the gastrointestinal tract of the living body and provided without an electronic component and a piezoelectric transducer.

FIG. 8 is a circuit diagram illustrating an example of a circuit configuration of the electronic component applicable to the medical mobile robot for the digestive system according to one exemplary embodiment of the present disclosure.

FIG. 9 is a graph illustrating an example of an ultrasonic input signal input to the piezoelectric transducer of the medical mobile robot and an output signal output from the piezoelectric transducer according to one exemplary embodiment of the present disclosure.

FIG. 10 is a graph illustrating an example of signal modulation by the electronic component of the medical mobile robot according to one exemplary embodiment of the present disclosure.

FIG. 11 is a cross-sectional view illustrating an example of a bioadhesive layer applicable to the medical mobile robot for the digestive system according to one exemplary embodiment of the present disclosure.

FIGS. 12 and 13 are views illustrating a method of using a medical mobile robot for a digestive system according to one exemplary embodiment of the present disclosure.

FIG. 14 is a view illustrating a method of operating the medical mobile robot for the digestive system according to one exemplary embodiment of the present disclosure.

FIG. 15 is a view illustrating the method of operating the medical mobile robot for the digestive system according to one exemplary embodiment of the present disclosure.

FIG. 16 is a view illustrating an example of the forms of electric fields that can be generated during a treatment process using the medical mobile robot for the digestive system according to one exemplary embodiment of the present disclosure.

FIG. 17 is a graph illustrating the treatment benefit of tumor penetration in a disease treatment using the medical mobile robot according to one exemplary embodiment of the present disclosure.

FIG. 18 is a view illustrating results of simulation for evaluating the tumor penetration and saturation efficiency of a treatment agent in the disease treatment using the medical mobile robot according to one exemplary embodiment of the present disclosure.

FIG. 19 is a graph illustrating the results obtained by evaluating the characteristics of cancer cell death in the disease treatment using the medical mobile robot according to one exemplary embodiment of the present disclosure.

FIG. 20 is a view illustrating a metal ion cloud (on the left side) generated by the medical mobile robot and a cancer cell (on the right side) killed by the medical mobile robot in the disease treatment using the medical mobile robot according to one exemplary embodiment of the present disclosure.

### DETAILED DESCRIPTION OF THE INVENTION

Hereinafter, exemplary embodiments of the present disclosure will be described in detail with reference to the attached drawings.

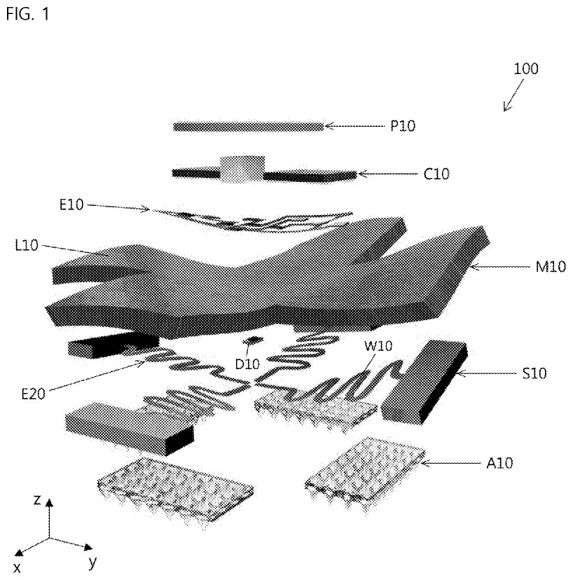

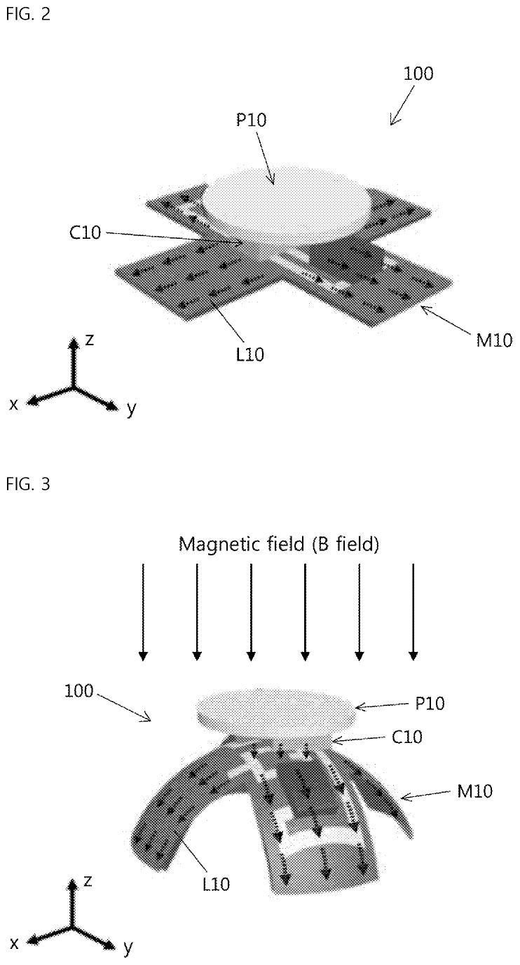

FIG. 1 is an exploded perspective view illustrating a medical mobile robot 100 for a digestive system according to one exemplary embodiment of the present disclosure. FIG. 2 is a perspective view illustrating the medical mobile robot 100 for the digestive system according to one exemplary embodiment of the present disclosure.

Referring to FIGS. 1 and 2, the medical mobile robot 100 for the digestive system according to one exemplary embodiment of the present disclosure may be a robot that is inserted into the digestive system of a living body to treat a disease or perform a medical procedure. The living body may be a human or an animal. The digestive system may encompass the entire digestive organ, including, but not limited to, the esophagus, stomach, duodenum, pancreas, small intestine, large intestine, etc. The medical mobile robot 100 may also be referred to as an ultra-small robot, mini robot, or micro robot. The medical mobile robot 100 may be a wireless robot or a remote-controlled robot.

The medical mobile robot 100 may include a magnetic body part M10, a piezoelectric transducer P10, an electronic component C10, and metal source members S10. In addition, the medical mobile robot 100 may further include a bioadhesive layer A10.

The magnetic body part M10 may serve as the body of the robot. The magnetic body part M10 may be configured to enable shape change and movement by a magnetic force within the digestive system. According to an example, the magnetic body part may include an elastic polymer matrix and a plurality of magnetic particles inserted into the elastic polymer matrix. The elastic polymer matrix may be or include, as a non-limiting example, a silicone rubber-based material [e.g., polydimethylsiloxane (PDMS), Ecoflex, etc.] or a polyurethane-based material. The plurality of magnetic particles may be or include, as a non-limiting example, NdFeB. The plurality of magnetic particles may be inserted into the elastic polymer matrix, and some of the magnetic particles may be present on the surface part of the elastic polymer matrix. The plurality of magnetic particles may have a magnetized state in a given direction. The magnetic body part M10 may have flexible characteristics. The magnetic body part M10 may be referred to as a kind of magnetic composite. However, the specific configuration of the magnetic body part M10 is not limited to that described above and may vary depending on cases. The thickness of the magnetic body part M10 may be, for example, about several tens of μm to several thousand μm.

The magnetic body part M10 may have a plurality of leg parts L10 extending outward from its central part in a fundamental form thereof. For example, the magnetic body part M10 may include three or more leg parts L10. The magnetic body part M10 may have a radial structure provided with three or more legs extending outward from its central part in its fundamental form. When the magnetic body part M10 includes three or more leg parts L10, the position of the magnetic body part M10 may be easily moved in any direction by using a magnetic field. In addition, the magnetic body part M10 may have a radial magnetization structure (i.e., a magnetization pattern) in which a magnetization direction of each of the plurality of leg parts L10 in the fundamental form is aligned outward from its central part (see FIG. 2). The magnetization pattern may be formed in each of the plurality of leg parts L10 so that the N pole or S pole faces outward. In this case, it may be easy to fold or unfold the plurality of leg parts L10 in one direction by using the magnetic field, and it may be easy to perform a predetermined operation. Meanwhile, the central part of the magnetic body part M10 may be magnetized in a direction parallel to the z-axis, i.e., in a vertical direction, in the fundamental form. The N or S pole may be arranged to face downwards in the central part (see FIG. 3).

According to an example, the magnetic body part M10 may have a cross-shaped structure when viewed from above in the fundamental form. In other words, from its central part, the magnetic body part M10 may have a structure in which four leg parts L10 extend in their four directions. In this case, when the magnetic body part M10 is moved within the digestive system by using a magnetic field, the magnetic body part M10 may be easily moved in a direction parallel to the x-axis, in a direction parallel to the y-axis, and in any direction within the xy plane. The magnetic field may be applied from outside a living body to inside the living body where the magnetic body part M10 exists. However, the magnetic body part M10 does not necessarily have to have the cross-shaped structure, and may have various shapes that can be changed.

The piezoelectric transducer P10 may be directly or indirectly coupled to the magnetic body part M10. The piezoelectric transducer P10 may perform the function of converting an ultrasonic signal into an electrical signal. The ultrasound signal may be a focused ultrasound signal. The ultrasonic signal may be applied from outside the living body to inside the living body where the piezoelectric transducer P10 exists, and the piezoelectric transducer P10 may convert the ultrasonic signal into the electrical signal. The electrical signal may be used to operate the medical mobile robot 100.

The electronic component C10 may be directly or indirectly coupled to the magnetic body part M10 and electrically connected to the piezoelectric transducer P10. The electronic component C10 may have a configuration for at least modulating or processing an electrical signal generated by the piezoelectric transducer P10. However, the function of the electronic component C10 is not limited to the modulating or processing of the electrical signal generated from the piezoelectric transducer P10, and may be configured to perform various electrical functions and/or roles as required.

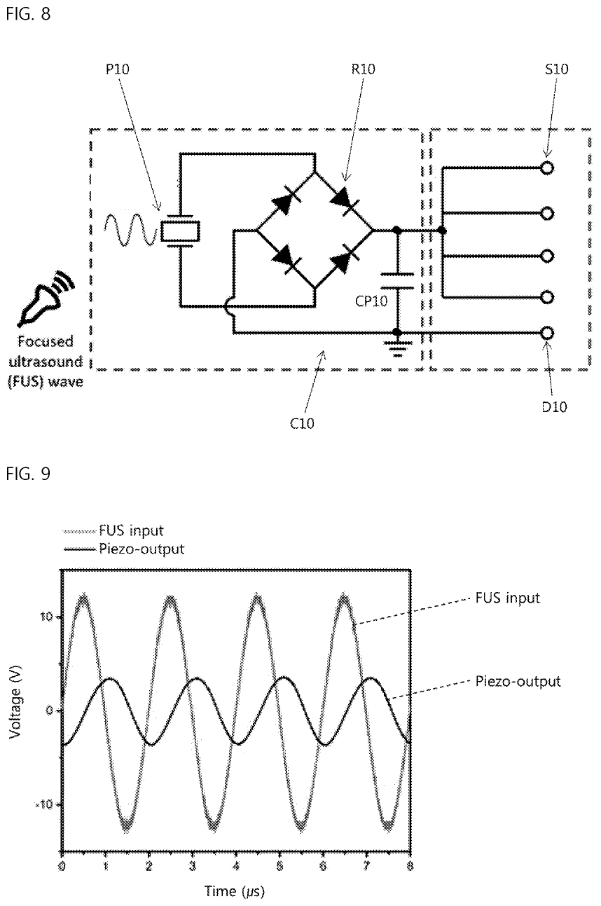

According to an example, the electronic component C10 may include a signal converter for converting an AC voltage signal into a DC voltage signal. The electrical signal generated from the piezoelectric transducer P10 may be the AC voltage signal, and the electronic component C10 may serve to convert the AC voltage signal into a DC voltage signal. The DC voltage signal may be used to emit metal ions from each metal source member S10. An exemplary configuration that the electronic component C10 may have will be described later with reference to FIG. 8.

Each metal source member S10 may be directly or indirectly coupled to the magnetic body member M10 and electrically connected to the electronic component C10. Each metal source member S10 may be a solid-state metal source capable of emitting metal ions by an electrical input applied from the electronic component C10. Each metal source member S10 may also be referred to as a metal storage member.

Each metal source member S10 may be composed entirely of metal or may be composed entirely of metal in an amount of 90 wt % or more or 95 wt % or more. Each metal source member S10 may be, for example, a bulk type metal body or a bulk-like metal body. The size of each metal source member S10 may be custom designed so as to suit a required dosage (i.e., an administration amount). As a non-limiting example, each metal source member S10 may satisfy at least one size condition among conditions of a length (a width) in the range of about 10 μm to 5 mm, a width (a height) in the range of about 10 μm to 1 mm, and a thickness in the range of about 100 nm to 1 mm. The thickness of each metal source member S10 may be in the range of about 100 nm to 500 μm, in the range of about 100 nm to 100 μm, or in the range of about 1 μm to 100 μm. In the use of each metal source member S10, a more useful thickness range may be in the range of about 100 nm to 100 μm or in the range of about 1 μm to 100 μm. However, the size conditions of each metal source member S10 are not limited to that described above and may vary as required.

Each metal source member S10 may be configured to emit metal ions by electrochemical dissolution. That is, in an environment where a certain solution exists inside the digestive system of the living body, each metal source member S10 may be electrochemically dissolved in response to an electrical input applied from the electronic component C10, thereby enabling the metal ions to be emitted. The medical mobile robot 100 may emit a large amount of metal ions from each metal source member S10 in an active and electrochemical manner. Here, the metal ions may include, for example, Cu²⁺ ions. The Cu²⁺ ions may be used to kill cancer cells through cuproptosis, which is the cell death mechanism, and be used to treat cancer. In this regard, each metal source member S10 may be a metal body composed of copper Cu. Each metal source member S10 may be composed entirely of Cu or may be composed of 90 wt % or more or 95 wt % or more of Cu.

The metal source members S10 may be arranged to be combined with the plurality of respective leg parts L10 of the magnetic body member M10. Accordingly, there is provided a plurality of metal source members S10, and the plurality of metal source members S10 may be arranged and coupled to the plurality of respective leg parts L10. A metal source member S10 may be placed on the lower surface of each leg part L10, and may be placed at a distal end (a terminal part) or at an area adjacent to the distal end on the lower surface of each leg part L10. However, the number and position of the metal source members S10 may vary depending on the cases.

The medical mobile robot 100 may include a first electrode member E10 and a second electrode member E20. For example, the first electrode member E10 may be placed on the upper surface side of the magnetic body part M10, and the second electrode member E20 may be placed on the lower surface side of the magnetic body part M10. In this case, the first electrode member E10 may be referred to as an upper electrode (i.e., an upper electrode structure), and the second electrode member E20 may be referred to as a lower electrode (i.e., a lower electrode structure). The first electrode member E10 and the second electrode member E20 may be electrically connected to each other. For example, although not illustrated in FIG. 1, the first and second electrode members E10 and E20 may be interconnected by a via conductor (a wire) formed to penetrate the magnetic body part M10. In addition, the first and second electrode members E10 and E20 may be electrically connected to the electronic component C10.

According to an example, the second electrode member E20 may include an electrode wiring element W10 connected to a metal source member S10. The electrode wiring element W10 may be placed on the lower surface of the magnetic body part M10. The electrode wiring element W10 connected to the metal source member S10 may be provided on each of the plurality of leg parts L10. A plurality of electrode wiring elements W10 may be provided, and each electrode wiring element W10 may have, for example, a winding shape. When the electrode wiring element W10 has the winding shape, it is able to act more flexibly in response to the shape change of the magnetic body part M10. In addition, the electrode wiring element W10 may serve to improve the features of shape change and movement (walking) of the magnetic body part M10 due to a magnetic field by controlling the characteristics such as the bending rigidity of the magnetic body part M10. The second electrode member E20 may be formed of, for example, copper (Cu) or other conductive material.

Additionally, the medical mobile robot 100 may further include a low voltage terminal D10. The low voltage terminal D10 may be placed on the lower surface side of the magnetic body part M10. The low voltage terminal D10 may be placed, for example, in the central part or an adjacent part thereto of the lower surface of the magnetic body part M10. The low voltage terminal D10 may be electrically connected to each electronic component C10 through the via conductor (the wire) penetrating the magnetic body part M10. When the medical mobile robot 100 is operated in a predetermined manner, a relatively low voltage may be applied to the low voltage terminal D10, and the relatively high voltage may be applied to each metal source member S10. The low voltage may be a ground voltage, a negative (−) voltage, or a positive (+) voltage. The high voltage may be the positive (+) voltage. Metal ions can be emitted from each metal source member S10 due to a potential difference that occurs between the low voltage terminal D10 and each metal source member S10 inside the digestive system. However, the position and shape of the low-voltage terminal D10 are not limited to the above description and that illustrated in FIG. 1, and may vary depending on the cases.

The first and second electrode members E10 and E20 may be considered as components included in the electronic component C10. In other words, the electronic component C10 may be considered to include the first and second electrode members E10 and E20. In addition, the low voltage terminal D10 may also be considered as a component included in the electronic component C10.

According to an example, the piezoelectric transducer P10 may be placed on the upper surface of the magnetic body layer M10, and the electronic component C10 may be placed between the magnetic body layer M10 and the piezoelectric transducer P10. The first electrode member E10 may be placed between the magnetic body layer M10 and the electronic component C10. Each metal source member S10 and the second electrode member E20 may be arranged on the lower surface of the magnetic body layer M10. However, the arrangement relationship of the components of the medical mobile robot 100 is not limited to that illustrated in FIG. 1 and may vary depending on the cases.

According to an example, other components, such as the second electrode member E20, other than each metal source member S10 on the lower surface side of the magnetic body layer M10, may be covered by a protective layer (not illustrated). The protective layer may be an insulating polymer layer. As the second electrode member E20 and the like are covered by the protective layer, the phenomenon of emitting metal ions from the second electrode member E20 may be prevented. Each metal source member S10 may be exposed without being covered by the protective layer, and metal ions may be emitted from each metal source member S10. Each metal source member S10 may serve as a type of electrode (terminal) in an electric circuit. In addition, the electronic component C10 and the first electrode member E10 on the upper surface of the magnetic body layer M10 may also be covered by a protective layer (not illustrated). When required, the piezoelectric transducer P10 may also be covered by a protective layer (not illustrated).

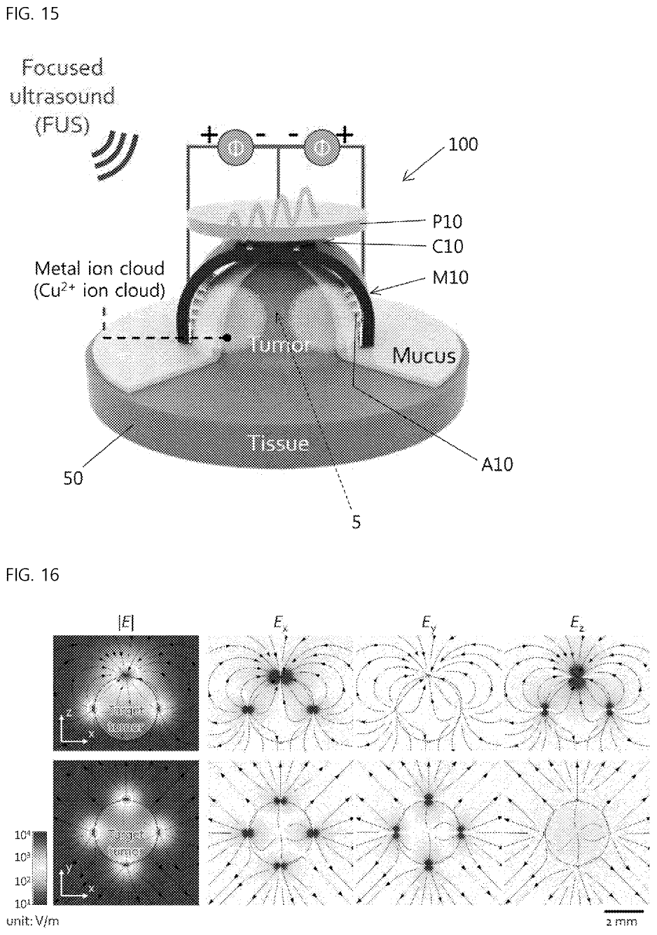

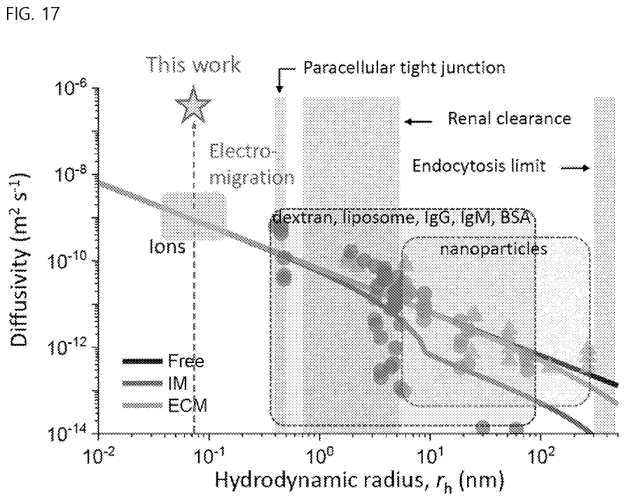

According to one exemplary embodiment of the present disclosure, the electronic component C10 and each metal source member S10 may be configured to generate electric fields while metal ions are emitted. For example, when the medical mobile robot 100 is operated in the predetermined manner, a relatively low voltage may be applied to the low voltage terminal D10, the relatively high voltage may be applied to each metal source member S10, and electric fields may be generated between each metal source member S10 and the low voltage terminal D10. Such electric fields may be applied directly to a target treatment site (a site of the living body) within a digestive system. The effective diffusivity of the metal ions to the target treatment site within the digestive system may be significantly increased by the electric fields. The increase in the effective diffusivity may be due to the electromigration effect caused by the electric fields. As a result, treatment effects such as the death of harmful cells by the metal ions may be significantly improved. This will be described in more detail later with reference to FIGS. 15 and 16.

The medical mobile robot 100 according to the exemplary embodiment may further include a bioadhesive layer A10 for increasing adhesive strength to the given living body site within the digestive system. The bioadhesive layer A10 may be placed on the lower surface of the medical mobile robot 100. For example, the bioadhesive layer A10 may be placed to cover at least a part of the electrode wiring element W10 while exposing each metal source member S10 on the lower surface of the magnetic body part M10. A bioadhesive layer A10 may be placed on the lower surface side of each of the plurality of leg parts L10 of the magnetic body part M10. Accordingly, a plurality of bioadhesive layers A10 may be provided. However, the number and arrangement of the bioadhesive layers A10 are not limited to that illustrated in FIG. 1 and may vary depending on the cases. The bioadhesive layer A10 may also be referred to as a bioadhesive pad.

The bioadhesive layer A10 may be configured to enable holding a targeted site of the living body well and adhering to the corresponding site of the living body well. The bioadhesive layer A10 may be configured to include: an elastic material layer having a plurality of protrusions on a surface thereof; and an adhesive coating layer placed to cover the plurality of protrusions on the elastic material layer. The specific configuration applicable to the bioadhesive layer A10 will be described in more detail later with reference to FIG. 11.

According to one exemplary embodiment, the medical mobile robot 100 may have a length in the range of about 0.5 to 20 mm and a width in the range of about 0.5 to 20 mm. In the fundamental form as illustrated in FIG. 2, the length of the medical mobile robot 100 in the x-axis direction may be about 0.5 to 20 mm or about 2 to 20 mm, and the width thereof in the y-axis direction may be about 0.5 to 20 mm or about 2 to 20 mm. Here, the terms “length” and “width” are just used for distinction, and the length can be referred to as the length in a first direction, and the width may be said to be the length in a second direction. The second direction may be a direction perpendicular to the first direction. In the medical mobile robot 100, the length and width may be the same with each other or different from each other. When the size of the medical mobile robot 100 is too small, it may be difficult to move it within a digestive system using a magnetic field. When the size of the medical mobile robot 100 is too large, it may be difficult to insert it into the digestive system of the living body. Accordingly, it may be advantageous for the medical mobile robot 100 to satisfy the size conditions described above. However, in some cases, at least one of the length and width of the medical mobile robot 100 may be outside the ranges described above.

According to one exemplary embodiment, the medical mobile robot 100 may be a cancer treatment robot for treating digestive system cancer. The medical mobile robot 100 may be configured to hold a site of a tumor (a cancer tumor) within the digestive system with the magnetic body part M10. In addition, the medical mobile robot 100 may be configured to emit a large amount and concentration of metal ions (e.g., Cu²⁺) from each metal source member S10 toward the tumor site in a state of holding the tumor site. In addition, the medical mobile robot 100 may be configured to increase the effective diffusivity of the metal ions to the tumor site by generating electric fields while the metal ions are emitted. Accordingly, the medical mobile robot 100 may perform cancer treatment in a method of precisely gripping the targeted tumor at its site and emitting a large amount of metal ions to the corresponding tumor site to kill the cancer cells.

FIG. 3 is a perspective view illustrating an example of a case in which a magnetic field is applied to the medical mobile robot for the digestive system, so as to change the shape of the magnetic body part according to one exemplary embodiment of the present disclosure. For convenience of description, each metal source member S10 and bioadhesive layer A10 in FIG. 1 are not illustrated in FIG. 3.

Referring to FIG. 3, the shape of the ‘magnetic body part M10 may be three-dimensionally changed by applying a magnetic field to the magnetic body part M10 of the medical mobile robot 100. In addition, the movement of the magnetic body part M10 may be easily controlled by controlling the direction and application position of the magnetic field applied to the magnetic body part M10. The shape of the medical mobile robot 100 inside the digestive system may be changed or its position may be moved by applying the magnetic field from outside the living body to the magnetic body part M10 while the medical mobile robot 100 is placed inside the digestive system. The strength of the magnetic field applied to the medical mobile robot 100 for shape change and movement (walking) of the medical mobile robot 100 may be, as a non-limiting example, about 100 mT or less or about 20 mT or less.

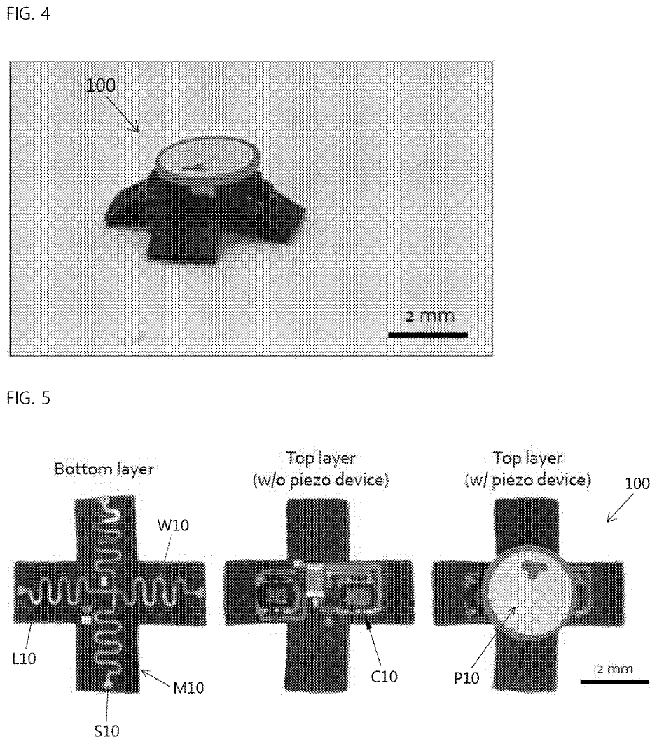

FIG. 4 is a photographic image showing the medical mobile robot 100 for the digestive system and manufactured according to one exemplary embodiment of the present disclosure.

FIG. 5 is a view showing photographic images of the lower and upper surfaces of the medical mobile robot 100 for the digestive system and manufactured according to one exemplary embodiment of the present disclosure. In FIG. 5, the leftmost image shows the lower surface of the medical mobile robot 100 with the bioadhesive layer excluded, the rightmost image shows the upper surface of the medical mobile robot 100, and the central image shows the upper surface thereof with the piezoelectric transducer P10 excluded. The medical mobile robot 100 in FIG. 5 may have a structure as that described with reference to FIGS. 1 and 2.

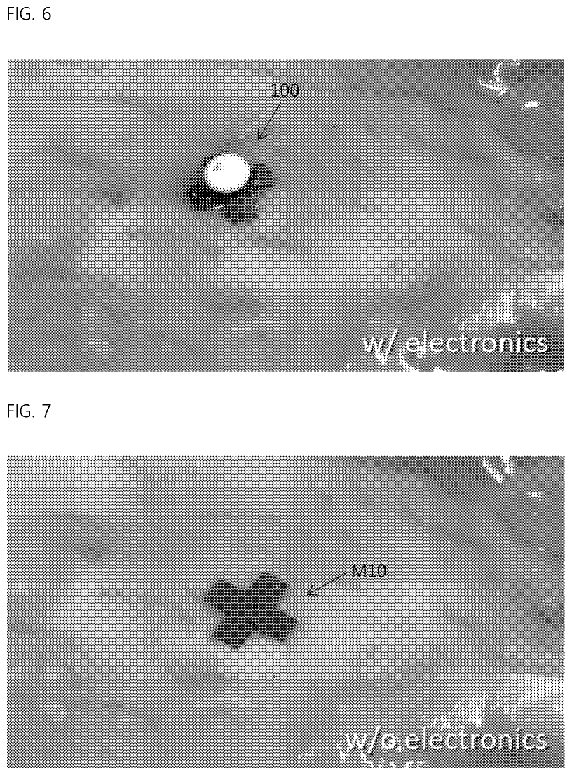

FIG. 6 is a photographic image showing a case in which the medical mobile robot 100 for the digestive system is positioned within a gastrointestinal tract of a living body according to one exemplary embodiment of the present disclosure.

Referring to FIG. 6, this photographic image shows a case where the medical mobile robot 100 is positioned inside a porcine stomach. By applying a magnetic field, the shape of the medical mobile robot 100 within a gastrointestinal tract may be changed and its position may be precisely moved to a desired place. When the electrode wiring element (W10 in FIG. 1) having the appropriate shape and size thereof is combined with the magnetic body part (M10 in FIG. 1) of the medical mobile robot 100, the rigidity of the magnetic body part (M10 in FIG. 1) increases and the characteristics such as bending stiffness are controlled, so that it is able to improve the characteristics of shape change and position movement of the medical mobile robot 100 within the gastrointestinal tract (in the digestive system). In other words, by combining the electronic component, etc., with the magnetic body part (M10 in FIG. 1), it is able to improve the characteristics of shape change and position movement of the medical mobile robot 100. In this regard, it is possible to implement the medical mobile robot 100 that is able to easily move (walk) in the desired direction and to the desired position and perform a given movement through shape change in an environment inside a digestive system of a living body, i.e., in an environment that is sticky, is humid (wet), has high friction, contains digestive fluid, mucus, or the like, and has movement (e.g., movement of the stomach).

The movement (walking) of the medical mobile robot 100 within the digestive system may be realized, for example, by rolling or tumbling. The movement (walking) method of the medical mobile robot 100 may be determined according to the geometric structure of the medical mobile robot 100, the magnetization pattern of the magnetic body part, and the application method of the magnetic field. In addition, in a case of having an appropriate size, the medical mobile robot 100 may move better within the digestive system.

FIG. 7 is a photographic image showing a case in which only the magnetic body part M10 is positioned within a gastrointestinal tract of the living body and provided without the electronic component and the piezoelectric transducer.

Referring to FIG. 7, this photographic image shows the case where only the magnetic body part M10 is positioned within the porcine colon without the electronic component and the piezoelectric transducer. In this case, compared to the exemplary embodiment in FIG. 6, it may be difficult to appropriately secure the rigidity and bending stiffness of the magnetic body part M10 without them, so it may not be relatively easy to secure excellent walking features and shape change features.

FIG. 8 is a circuit diagram illustrating an example of a circuit configuration of the electronic component applicable to the medical mobile robot for the digestive system according to one exemplary embodiment of the present disclosure.

Referring to FIG. 8, the medical mobile robot for the digestive system according to the exemplary embodiment of the present disclosure may include a piezoelectric transducer P10, an electronic component C10, and metal source members S10. The electronic component C10 may include, for example, rectifiers R10 and a capacitor CP10. Each rectifier R10 may be a diode. A plurality of rectifiers R10 can be connected one another so as to form a ring circuit structure. Each metal source member S10 may be connected to the ring circuit structure, and the capacitor CP10 may be connected between the ring circuit structure and the metal source members S10. The piezoelectric transducer P10 may be connected to the ring circuit structure. In addition, a low voltage terminal D10 connected to both the ring circuit structure and the capacitor CP10 may be further provided. The combination of each rectifier R10 and the capacitor CP10 may serve as a signal converter for converting an AC voltage signal into a DC voltage signal.

The piezoelectric transducer P10 may perform the function of converting an ultrasonic signal into an electrical signal. The ultrasound signal may be a focused ultrasound signal. The ultrasonic signal may be applied from outside the living body to inside the living body where the piezoelectric transducer P10 exists, and the piezoelectric transducer P10 may convert the ultrasonic signal into the electrical signal. The electrical signal generated from the piezoelectric transducer P10 may be an AC voltage signal, and the electronic component C10 may serve to convert the AC voltage signal into a DC voltage signal. The DC voltage signal may be used to emit metal ions from each metal source member S10. In addition, the electronic component C10 and each metal source member S10 may be configured to generate electric fields while the metal ions are emitted.

However, the specific configuration of the electronic component C10 described with reference to FIG. 8 is merely an example and may be changed in various ways.

FIG. 9 is a graph illustrating an example of an ultrasonic input signal (i.e., FUS input) input to the piezoelectric transducer of the medical mobile robot and an output signal (i.e., piezo-output) output from the piezoelectric transducer according to one exemplary embodiment of the present disclosure.

Referring to FIG. 9, the output signal (i.e., the piezo-output) output from the piezoelectric transducer by the ultrasonic input signal (i.e., the FUS input) may have an AC voltage form.

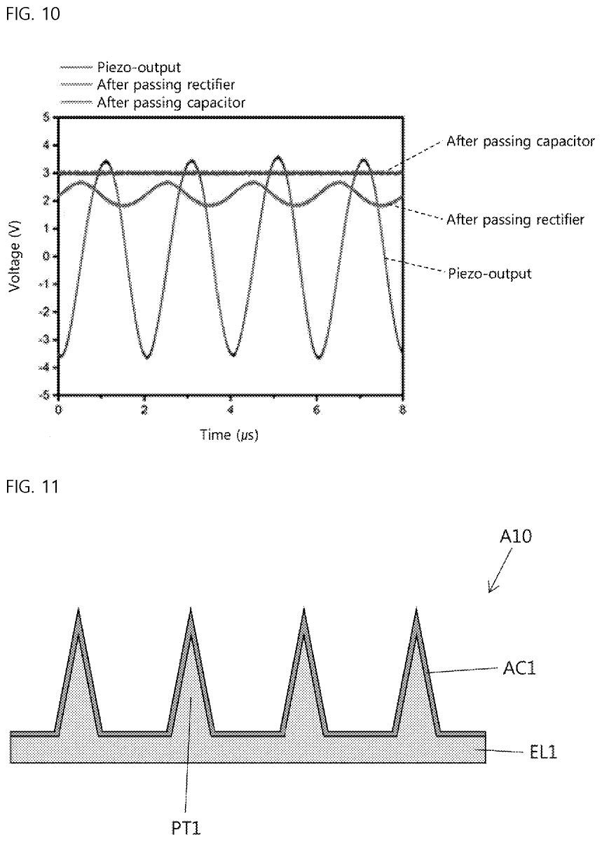

FIG. 10 is a graph illustrating an example of signal modulation by the electronic component of the medical mobile robot according to one exemplary embodiment of the present disclosure.

Referring to FIG. 10, the output signal (i.e., the piezo-output) output from the piezoelectric transducer may be modulated while passing through the rectifiers and capacitor of the electronic component. The output signal (i.e., the piezo-output) output from the piezoelectric transducer may be an AC voltage signal, and the AC voltage signal may be converted into a DC voltage signal by the electronic component. The DC voltage signal may be applied between each metal source member S10 and the low voltage terminal D10 in FIG. 8.

FIG. 11 is a cross-sectional view illustrating an example of a bioadhesive layer A10 applicable to the medical mobile robot for the digestive system according to one exemplary embodiment of the present disclosure.

Referring to FIG. 11, the bioadhesive layer A10 may include: an elastic material layer EL1 having a surface provided with a plurality of protrusions PT1 thereon; and an adhesive coating layer AC1 placed on the elastic material layer EL1 to cover the plurality of protrusions PT1. The elastic material layer EL1 may be composed of a certain polymer material. The plurality of protrusions PT1 may protrude in a direction perpendicular to the basal layer of the elastic material layer EL1 and may have, for example, a tapered shape in which its width becomes narrower toward the end thereof. The plurality of protrusions PT1 may have, for example, a cone shape or a shape similar to the same. The plurality of protrusions PT1 may be arranged two-dimensionally with a predetermined length (a height) and an interval. The plurality of protrusions PT1 may serve to hold a target site (a site of the living body) as it embeds itself to a certain extent into a tissue surface layer of the target site (the site of the living body) within the digestive system.

The adhesive coating layer AC1 may be placed to cover the plurality of protrusions PT1 on the elastic material layer EL1. The adhesive coating layer AC1 may be composed of a material that adheres well to the site of the living body (i.e., the biological tissue). As a non-limiting example, the adhesive coating layer AC1 may include a hydrogel. The hydrogel may include a backbone material and a functional group bonded to the same. Within the tissue of the living body, functional groups such as amine, carboxylic acid, thiol, and imidazole may exist, and particularly, there may exist a lot of amine groups. The hydrogel may have the property of adhering (bonding) well to the functional groups such as the amine groups of the tissue. Accordingly, the hydrogel may adhere well to mucus and the like inside the digestive system. The hydrogel may include, as a non-limiting example, at least one of chitosan, alginate, hyaluronic acid, polyacrylic acid, polyacrylamide, and polyethyleneimine. However, the specific configuration of the adhesive coating layer AC1 is not limited to that described above and may vary depending on the cases.

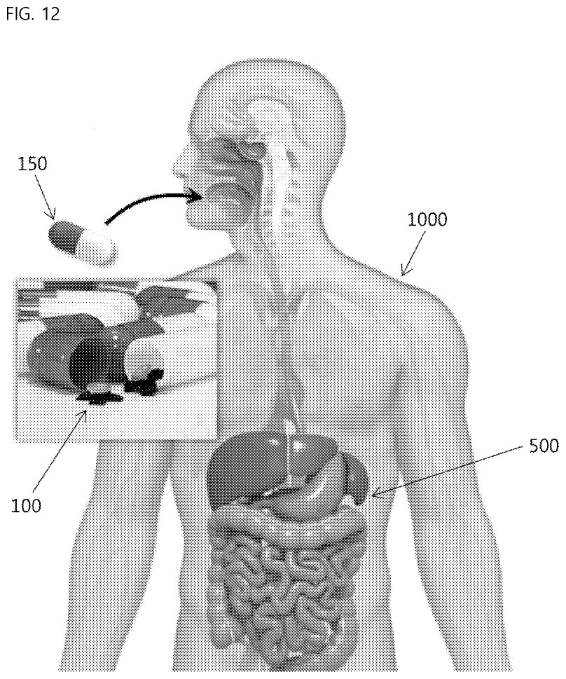

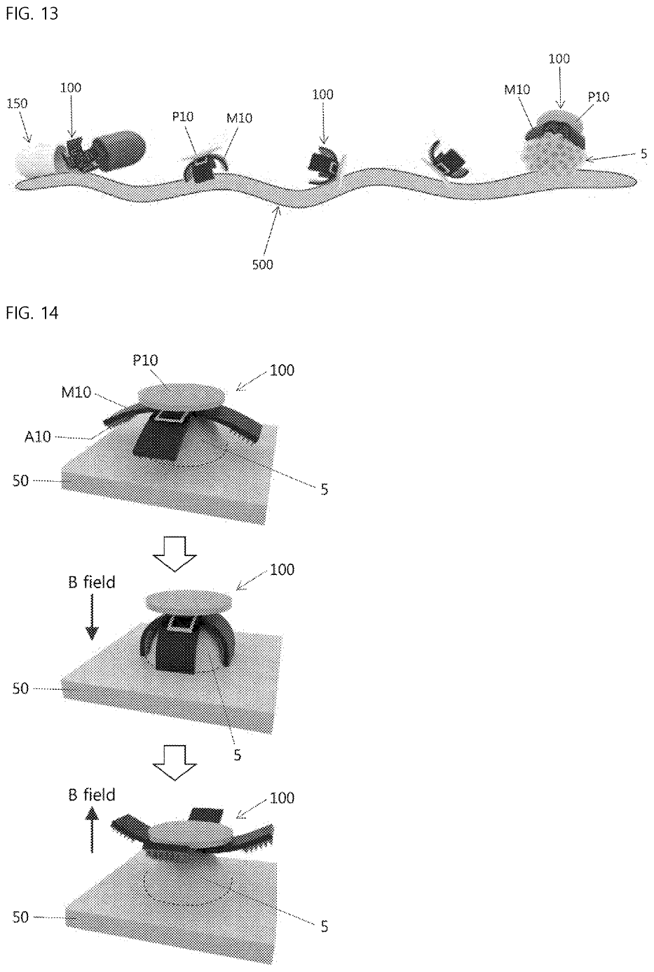

FIGS. 12 and 13 are views illustrating a method of using a medical mobile robot for a digestive system according to one exemplary embodiment of the present disclosure.

Referring to FIGS. 12 and 13, the method of using the medical mobile robot 100 according to one exemplary embodiment of the present disclosure may include: inserting the medical mobile robot 100 into a digestive system 500 of a living body 1000; and operating the medical mobile robot 100 within the digestive system 500 of the living body 1000. The medical mobile robot 100 may have a configuration as that described with reference to FIGS. 1 and 2. The medical mobile robot 100 may include a magnetic body part M10, a piezoelectric transducer P10, an electronic component C10, and metal source members S10. In addition, the medical mobile robot 100 may further include a bioadhesive layer A10.

The medical mobile robot 100 may be inserted into the digestive system 500 of the living body 1000 in a method of oral administration. For example, the medical mobile robot 100 may be inserted into a gastrointestinal tract of a human body in a manner that a person ingests it through the mouth (i.e., the method of oral administration). In a state of being placed in a capsule 150, the medical mobile robot 100 may be administered into the living body 1000 in the method of oral administration. Within the specific digestive system 500, at least a part of the capsule 150 can be dissolved or the medical mobile robot 100 can come out of the capsule 150. This capsule 150 is able to be melt in a predetermined gastrointestinal site depending on its composition material, and accordingly, the medical mobile robot 100 can come out of the capsule 150 in the corresponding gastrointestinal site.

The operating of the medical mobile robot 100 may include: changing a shape or moving a position of the medical mobile robot 100 within the digestive system 500; and emitting metal ions from each metal source member (S10 in FIG. 1). In another aspect, the operating of the medical mobile robot 100 may include: applying a magnetic field to the medical mobile robot 100; and applying an ultrasonic signal to the medical mobile robot 100.

The changing of the shape or moving of the position of the medical mobile robot 100 within the digestive system 500 may be performed by applying a magnetic field to the medical mobile robot 100. The magnetic field is applied from outside the living body 1000 to the magnetic body part M10 while the medical mobile robot 100 is placed inside the digestive system 500, whereby the shape of the medical mobile robot 100 inside the digestive system 500 is able to be changed or its position may be moved. The direction and application position of the magnetic field applied to the magnetic body part M10 may adjusted, whereby the movement of the medical mobile robot 100 may be easily controlled. The position of the medical mobile robot 100 within the digestive system 500 may be precisely and accurately controlled. In addition, the magnetic body part M10 may be operated so as to hold a target treatment site (a site of the living body) within the digestive system 500. The target treatment site may be, for example, a tumor site 5. The tumor site 5 may be a cancer tumor.

The emitting of the metal ions from each metal source member (S10 in FIG. 1) may be performed by applying an ultrasonic signal to the medical mobile robot 100. For example, the ultrasonic signal is applied from outside the living body 1000 to the piezoelectric transducer P10 while the tumor site 5 is held with the magnetic body part M10, whereby the metal ions can be emitted from each metal source member (S10 in FIG. 1). The metal ions may include, for example, Cu²⁺ ions. Since each metal source member (S10 in FIG. 1) can be a bulk type metal body or a bulk-like metal body, the metal source member S10 may emit a large amount of metal ions to the tumor site 5. The metal ions may serve to kill cancer cells in the tumor site 5 according to the cuproptosis mechanism.

The operating of the medical mobile robot 100 may be performed while capturing an image of the digestive system 500. That is, the medical mobile robot 100 may be operated while capturing the image of the digestive system 500. For example, the imaging of the digestive tract (500) can be performed by using ultrasound or endoscopic imaging, and the operations may be performed while the positions of the medical mobile robot 100 and the target site are checked.

FIG. 14 is a view illustrating a method of operating a medical mobile robot for a digestive system according to one exemplary embodiment of the present disclosure.

Referring to FIG. 14, as in the first and second stages in FIG. 14, a magnetic field in a first direction is applied to the magnetic body part M10 while the medical mobile robot 100 is placed on the tumor site 5 in the digestive system, whereby the magnetic body part M10 may grip the tumor site 5. That is, the tumor site 5 may be held by the plurality of leg parts of the magnetic body part M10. At this time, the bioadhesive layer A10 may be adhered to the tumor site 5 with relatively strong adhesive strength.

When required, as in the third stage of the FIG. 14, a magnetic field in a second direction is applied to the magnetic body part M10, whereby a reverse operation of the second stage is able to be performed. In other words, the magnetic body part M10 may release the tumor site 5 that it is holding. The second direction may be the opposite direction to the first direction. In FIG. 14, reference numeral 50 represents an inner wall tissue of the digestive system.

FIG. 15 is a view illustrating the method of operating the medical mobile robot for the digestive system according to one exemplary embodiment of the present disclosure. For convenience of description, a part of the medical mobile robot and a part of the mucus are omitted in FIG. 15.

Referring to FIG. 15, as illustrated in the second stage in FIG. 14, in a state of holding the tumor site 5 by the magnetic body part M10 of the medical mobile robot 100, metal ions may be emitted from each metal source member (S10 of FIG. 1). The emitting of the metal ions from each metal source member (S10 in FIG. 1) may be performed by applying an ultrasonic signal to the medical mobile robot 100. The ultrasonic signal (e.g., the focused ultrasonic signal) is applied from outside the living body to the piezoelectric transducer P10, whereby the metal ions may be emitted from each metal source member (S10 in FIG. 1). The emitting of the metal ions may be performed actively and electrochemically. The metal ions may include, for example, Cu²⁺ ions. Since each metal source member (S10 in FIG. 1) may be a bulk type metal body or a bulk-like metal body, each metal source member S10 may emit a large amount of metal ions to the tumor site 5. The metal ions may be emitted in large quantities, forming a metal ion cloud on and near the tumor site 5. The metal ions may serve to intensively kill cancer cells in the tumor site 5 according to the cuproptosis mechanism.

In addition, according to the exemplary embodiment, the electronic component C10 and each metal source member (S10 of FIG. 1) of the medical mobile robot 100 may be configured to generate electric fields while metal ions are emitted. For example, as described above, when the medical mobile robot 100 is operated in the predetermined manner, a relatively low voltage may be applied to the low voltage terminal (D10 of FIG. 1), a relatively high voltage may be applied to each metal source member (S10 of FIG. 1), and electric fields may be generated between each metal source member (S10 of FIG. 1) and the low voltage terminal (D10 of FIG. 1). Such electric fields may be applied directly to the tumor site 5. The effective diffusivity of the metal ions to the tumor site 5 may be significantly increased by the electric fields. The increase in the effective diffusivity may be due to the electromigration effect caused by the electric fields. As a result, treatment effects such as the death of cancer cells by the metal ions may be significantly improved.

The method of using and operating the medical mobile robot described with reference to FIGS. 12 to 15 may correspond to a treatment method or a surgical procedure for a disease in a digestive system by using the medical mobile robot. Accordingly, the method for treating or surgically treating the disease in the digestive system according to the exemplary embodiments of the present disclosure may include the stages and features described with reference to FIGS. 12 to 15.

The conventional targeted anticancer therapy is administered by injecting an anticancer drug or treatment agent into a living body through intravascular injection, such as an intravenous injection. However, in this case, there is a problem that the accuracy and efficiency of drug delivery are significantly reduced. The probability that the drug injected into the bloodstream will reach a cancer tumor is less than 1%, and even though the drug reaches the tumor site, the drug has to penetrate into the tumor through diffusion, so the probability that the drug will enter the inside of the tumor may be less than 0.001%. To increase treatment efficacy, the concentration and amount of the treatment agent injected into the bloodstream have to be increased, but this is limited owing to lethal doses and side effects. The overdose of the drug in the bloodstream can cause the side effects at non-target sites. Even in a treatment method case that delivers nanoparticles conjugated with copper ions by using the intravenous injection, a drug dosage may be very limited and the drug delivery efficiency may also be extremely low.

However, according to the exemplary embodiment of the present disclosure, the medical mobile robot for the digestive system and the method of using the same is implementable, the medical mobile robot being inserted into the digestive system of a living body to treat a disease or perform a medical procedure, and being able to dramatically improve the delivery efficiency and delivery accuracy of a treatment substance to a target site. In addition, according to the exemplary embodiment of the present disclosure, the medical mobile robot and the method of using the same is implementable, the medical mobile robot being able to easily move (walk) in a desired direction and to a desired position and perform a given movement through shape change in an environment inside a digestive system of a living body, i.e., in an environment that is sticky, is humid (wet), has high friction, contains digestive fluid, mucus, or the like, and has movement (e.g., the movement of the stomach). In addition, according to the exemplary embodiment of the present disclosure, the medical mobile robot and the method of using the same is implementable, the medical mobile robot being able to significantly improve the treatment effect of diseases such as digestive system cancer by positioning itself accurately at a target site within the digestive system of the living body to release a large amount of the treatment substance and dramatically improving the penetration efficiency of the treatment substance into the target. In particular, when each metal source members, which is the bulk type metal body or the bulk-like metal body, is inserted into the human body through the digestive system, a large amount of metal ions may be emitted intensively from each metal source member to the target site without problems such as lethal dose or side effects, so the penetration efficiency of the metal ions into the target may be greatly improved. Using the treatment method (the medical method) according to the exemplary embodiment of the present disclosure enables to improve the delivery efficiency of the treatment substance (the drug) to the target within the digestive system to almost 100%, significantly increase the treatment effect with a large amount of the treatment substance, and suppress or prevent the problem of side effects from occurring.

FIG. 16 is a view illustrating an example of the forms of electric fields that can be generated during a treatment process using the medical mobile robot for the digestive system according to one exemplary embodiment of the present disclosure.

Referring to FIG. 16, the medical mobile robot according to the exemplary embodiment of the present disclosure may emit metal ions while holding a target tumor site with the magnetic body part, and along with this, generate electric fields. The upper graphs in FIG. 16 illustrate the forms of the electric fields in the xz plane, and the lower graphs in FIG. 16 illustrate the forms of the electric fields generated in the xy plane. Ex represents each x-axis component of the electric fields, Ey represents each y-axis component of the electric fields, and Ez represents each z-axis component of the electric fields. Meanwhile, |E| represents the electric fields corresponding to absolute values.

FIG. 17 is a graph illustrating the treatment benefit of tumor penetration in the disease treatment using the medical mobile robot according to one exemplary embodiment of the present disclosure. FIG. 17 shows hydrodynamic radius versus diffusivity of treatment agents within a tumor environment. Here, the diffusivity may refer to the inherent diffusion characteristics of the treatment agents within a given medium. In FIG. 17, ECM represents an extracellular matrix, IM represents an interstitial matrix, and Free represents a water or physiological fluid environment.

Referring to FIG. 17, the medical mobile robot according to the exemplary embodiment of the present disclosure may be advantageous in securing high diffusivity due to using metal ions as a type of treatment agent. The diffusion coefficient may be inversely proportional to the size of treatment agent particle. That is, the smaller the size of a particle, the better the particle can diffuse. The size of typical anticancer drug may be as small as a few nanometers (nm), and the size of nanoparticle may be approximately 50 to 100 nm. Meanwhile, the size of copper ion may be about 0.065 nm. Due to such size differences, when the metal ions (e.g., the copper ions) are used, it is possible to secure diffusivity that is approximately 10² to 10³ times higher than that of the general nanoparticles.

In addition, in the present exemplary embodiment of the present disclosure, instant and significant increase in the local concentration of metal ions may be achievable in the target site. The medical mobile robot according to the exemplary embodiment may electrochemically emit a large amount of metal ions (e.g., copper ions), and may instantaneously increase the local concentration of the metal ions by about 10³ to 10⁴ times. A diffusion flux may be created proportional to a concentration gradient. Accordingly, the effective flux of the treatment agent to the target site (e.g., the tumor) may be increased by approximately 10³ to 10⁴ times.

In addition, in the exemplary embodiment of the present disclosure, electric fields are generated while metal ions are emitted, whereby the effective diffusivity of the metal ions may be improved by about 10² times due to the electromigration effect caused by the electric fields. The metal ions may be strongly attracted into the cancer tumor by the electric fields.

Consequently, according to the above-described exemplary embodiment of the present disclosure, the penetration efficiency and treatment efficacy of the treatment agent into the target site (the tumor site) may be improved by up to 10⁸ to 10⁹ times compared to the comparative example or the conventional method.

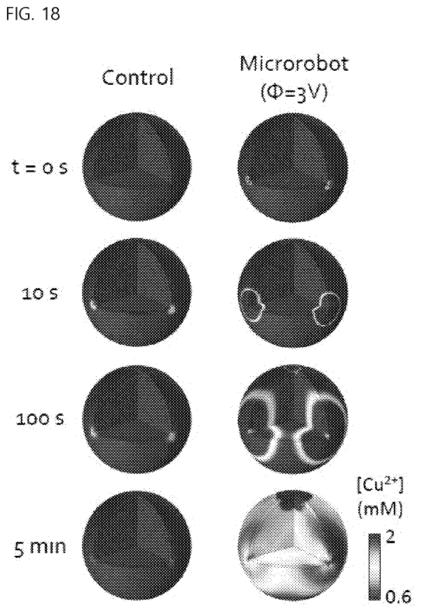

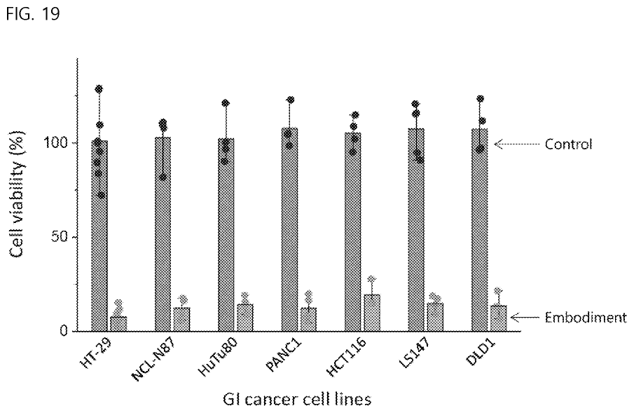

FIG. 18 is a view illustrating results of simulation for evaluating the tumor penetration and saturation efficiency of the treatment agent in the disease treatment using the medical mobile robot according to one exemplary embodiment of the present disclosure. FIG. 18 includes the results of the embodiment (Microrobot) and the results of the control group (Control).

Referring to FIG. 18, according to the exemplary embodiment of the present disclosure, this view shows that the medical mobile robot makes the results of saturating the inside of the tumor with metal ions (Cu²⁺) within about 5 minutes after emitting the metal ions, and on the other hand, in the control group, it can be confirmed that the treatment agent barely penetrates into the tumor.

FIG. 19 is a graph illustrating the results of evaluating the characteristics of cancer cell death in the disease treatment using the medical mobile robot according to one exemplary embodiment of the present disclosure. FIG. 19 includes the results of the embodiment (Embodiment) and the results of the control group (Control). The exemplary embodiment corresponds to a case where the medical mobile robot is used to hold a tumor site and perform a treatment for 30 minutes at a voltage of 3 V.

Referring to FIG. 19, it can be confirmed that when the medical mobile robot according to the exemplary embodiment of the present disclosure is used, most digestive cancer cells, i.e., gastrointestinal cancer cells (GI cancer cells), can be killed in a very short period of time.

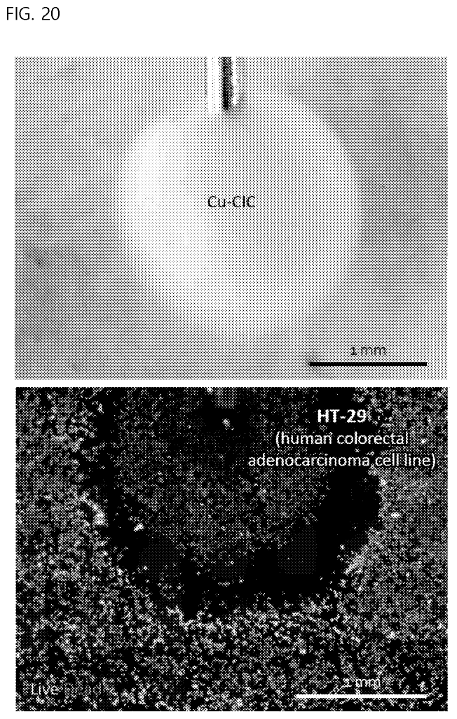

FIG. 20 is a view illustrating both a metal ion cloud, i.e., a Cu²⁺ ion cloud CIC, (on the left side) generated by the medical mobile robot and a cancer cell (on the right side) killed by the medical mobile robot in the disease treatment using the medical mobile robot according to one exemplary embodiment of the present disclosure.

According to the exemplary embodiments of the present disclosure described above, the medical mobile robot for the digestive system and the method of using the same is implementable, the medical mobile robot being inserted into the digestive system of the living body to treat a disease or perform a medical procedure, and being able to dramatically improve the efficiency and accuracy of delivering the treatment substance to the target site. In addition, according to the exemplary embodiments of the present disclosure, the medical mobile robot and the method of using the same is implementable, the medical mobile robot being able to easily move (walk) in the desired direction and to the desired position and perform the given movement through the shape change in the environment inside the digestive system of the living body, i.e., in the environment that is sticky, is humid (wet), has high the friction, contains the digestive fluid, mucus, or the like, and has the movement (e.g., the movement of the stomach). In addition, according to the exemplary embodiments of the present disclosure, the medical mobile robot and the method of using the same is implementable, the medical mobile robot being able to significantly improve the treatment effect of the diseases such as digestive system cancer by positioning itself accurately at the target site within the digestive system of the living body to release a large amount of the treatment substance and dramatically improving the penetration efficiency of the treatment substance into the target. Using the treatment method (the medical method) according to the exemplary embodiments of the present disclosure enables to improve the delivery efficiency of the treatment substance (the drug) to the target within the digestive system to almost 100%, significantly increase the treatment effect with a large amount of the treatment substance, and suppress or prevent the problem of side effects from occurring.

Additionally, the medical mobile robot according to the exemplary embodiment of the present disclosure may also be used for the disease treatment other than cancer. The medical mobile robot may be used to perform the disease treatments or medical measures within the digestive system.

In addition, the medical mobile robot according to the exemplary embodiment of the present disclosure may be used for implanting (delivering) an electronic component into a living body. When the medical mobile robot is used, the electronic component can be implanted (delivered) accurately and precisely to a desired position in a digestive system without surgical procedures such as open surgery or laparoscopic surgery. When the medical mobile robot is used for implanting (delivering) the electronic component, the corresponding medical mobile robot may or may not include metal source members. Therefore, in this case, the medical mobile robot may include a magnetic body, a piezoelectric transducer, and an electronic component, or may include a magnetic body, a piezoelectric transducer, an electronic component, and metal source members. In addition, the medical mobile robot may further include a bioadhesive layer.

What is claimed is:

1. A medical mobile robot for a digestive system and being inserted into the digestive system of a living body to treat a disease or perform a medical procedure, the medical mobile robot comprising:
a magnetic body part configured to enable shape change and movement within the digestive system due to magnetic force;
a piezoelectric transducer coupled to the magnetic body part to convert an ultrasonic signal into an electrical signal;
an electronic component coupled to the magnetic body part to at least modulate or process the electrical signal generated by the piezoelectric transducer; and
solid-state metal source members coupled to the magnetic body part and capable of emitting metal ions in response to an electrical input from the electronic component.

2. The medical mobile robot of claim 1, wherein the magnetic body part comprises:
an elastic polymer matrix; and
a plurality of magnetic particles inserted into the elastic polymer matrix.

3. The medical mobile robot of claim 1, wherein the magnetic body part has a radial structure provided with three or more leg parts extending outward from a central part thereof when viewed from above in a fundamental form.

4. The medical mobile robot of claim 1, wherein the magnetic body part is provided with a plurality of leg parts extending outward from a central part thereof in a fundamental form, and
the magnetic body part has a radial magnetization structure in which a magnetization direction of each of the plurality of leg parts is aligned outward from the central part.

5. The medical mobile robot of claim 1, wherein the electronic component comprises:
a signal converter for converting an AC voltage signal into a DC voltage signal.

6. The medical mobile robot of claim 1, wherein each metal source member is configured to emit the metal ions by electrochemical dissolution.

7. The medical mobile robot of claim 1, wherein each metal source member is a bulk-type metal body.

8. The medical mobile robot of claim 1, wherein each metal source member satisfies at least one size condition among conditions of a length in a range of 10 μm to 5 mm, a width in a range of 10 μm to 1 mm, and a thickness in a range of 100 nm to 1 mm.

9. The medical mobile robot of claim 1, wherein the metal ions comprise Cu²⁺ ions.

10. The medical mobile robot of claim 1, wherein the magnetic body part is provided with a plurality of leg parts extending outward from a central part thereof in a fundamental form, and
the metal source members are arranged to be combined with the plurality of respective leg parts.

11. The medical mobile robot of claim 10, wherein an electrode wiring element connected to each metal source member is provided on each of the plurality of leg parts, and
the electrode wiring element has a winding shape.

12. The medical mobile robot of claim 1, wherein the electronic component and each metal source member are configured to generate electric fields while the metal ions are emitted, and
effective diffusivity of the metal ions increases by the electric fields.

13. The medical mobile robot of claim 1, further comprising:
a bioadhesive layer for increasing adhesive strength to a given site of the living body within the digestive system.

14. The medical mobile robot of claim 13, wherein the bioadhesive layer comprises:
an elastic material layer having a surface provided with a plurality of protrusions thereon; and
an adhesive coating layer placed on the elastic material layer so as to cover the plurality of protrusions.

15. The medical mobile robot of claim 1, wherein the medical mobile robot has a length in a range of 0.5 to 20 mm and a width in a range of 0.5 to 20 mm.

16. The medical mobile robot of claim 1, wherein the medical mobile robot is a cancer treatment robot for treating digestive system cancer.

17. The medical mobile robot of claim 1, wherein the medical mobile robot is configured to hold a tumor site within the digestive system with the magnetic body part.

18. A method of using a medical mobile robot described in claim 1, the method comprising:
inserting the medical mobile robot into a digestive system of a living body; and
operating the medical mobile robot within the digestive system of the living body.

19. The method of claim 18, wherein the medical mobile robot is inserted into the living body in a method of oral administration in a state of being placed in a capsule, and
at least a part of the capsule is dissolved or the medical mobile robot comes out of the capsule within the predetermined digestive system.

20. The method of claim 18, wherein the operating of the medical mobile robot comprises:
applying a magnetic field to the medical mobile robot; and
applying an ultrasonic signal to the medical mobile robot.

21. The method of claim 18, wherein the operating of the medical mobile robot comprises:
changing a shape or moving a position of the medical mobile robot within the digestive system; and
emitting metal ions from each metal source member.

22. The method of claim 18, wherein the medical mobile robot is operated while capturing an image of the digestive system.
